(19) United States
(12) Plant Patent Application Publication
(10) Pub. No.: US 2007/0039080 P1
(43) Pub. Date: Feb. 15, 2007

(54) Mango tree named "Leopold"

(76) Inventor: William John Gabay, North Miami Beach, FL (US)

Correspondence Address: FLEIT KAIN GIBBONS GUTMAN BONGINI & BIANCO, 21355 EAST DIXIE HIGHWAY, SUITE 115, MIAMI, FL, 33180, US

(21) Appl. No.: 11/199,894

(22) Filed: Aug. 9, 2005

(51) Int. Cl. A01H 5/00

(52) U.S. Cl. PLT/159

(57) ABSTRACT

The present invention provides a new and distinct variety of mango. The new mango tree, named “Leopold,” produces fruit distinguishable from other mango varieties by its quality of flesh which includes only a small amount of fiber attached to the husk of the freestone seed. The average weight of the mango fruit is about 13-18 ounces when mature and has a smooth skin which is yellow-gold and red when ripe. The new mango fruit has good storage and handling qualities.

Genus species: Mangifera indica. L.

Denomination: Leopold.

### BACKGROUND OF THE INVENTION

A mango tree is a medium to large (about 30 to 90 feet) evergreen with a symmetrical, rounded canopy ranging from low and dense to upright and open. The bark of the mango tree is usually dark grey-brown to black, generally smooth, and superficially cracked or fissured. The mango tree forms a long unbranched long taproot plus a dense mass of superficial feeder roots. In deep soil, the taproot descends to a depth of about 20 feet. The profuse, wide-spreading feeder roots also send many anchor roots which penetrate for several feet.

The leaves of the mango tree may be about 4 to 12 inches in length and ¾ to 2 inches in width. The leaves may have a variety of shapes such as oval-lanceolate, lanceolate, oblong, linear-oblong, ovate, obovate-lanceolate, or roundish-oblong. Hermaphrodite and male flowers are produced in the same panicle. The flowers are usually yellowish or reddish in color and are borne in profuse, showy, erect, pyramidal, branched clusters in the fruits. The size of both the male and hermaphrodite flowers varies from about ¼ to ½ inches in diameter.

The mango fruit is generally a compressed, fleshy drupe. It varies considerably in size, shape, color, fiber content, flavor, and taste. The fruit may be nearly round, oval, ovoid-oblong, or somewhat kidney-shaped, and is usually more or less lop-sided. The fruit ranges from 2½ to 10 inches in length and from a few ounces to 4 or 5 pounds. The skin of the fruit is leathery, waxy, smooth, fairly thick, aromatic and ranges from light- or dark-green to clear yellow, yellow-orange, yellow and reddish-pink, or generally blushed with bright- or dark-red or purple-red, with fine yellow, greenish or reddish dots, and thin or thick whitish, gray, or purplish bloom, when fully ripe. The most characteristic feature of the fruit is the formation of a small conical projection developing laterally at the proximal end of the fruit, known as the beak. The beak may be prominent in some, less in others, and in some varieties it is represented merely as a dot.

### SUMMARY OF THE INVENTION

The present invention relates to a new variety of tropical mango which was discovered in North Miami Beach, Fla. The fruit of the discovered mango tree is characterized as having a similar aroma, flavor, taste, texture of flesh, and lack of fiber as the variety of mango named, Bombay Green. Relative to the Bombay Green, the mango of the present invention has better keeping qualities. In other words, the mango of the present invention has an extended shelf life, a brightly colored fruit, a resistance to bruising, a resistance to wind, and a degree of resistance to anthracnose. The new mango tree produces medium red fruit on a yellow ground color, and matures in July and August in North Miami Beach, Fla. The fruit as described usually falls to the ground at maturity and ripens in 5-10 days at 75° to 80°. The fruit can be held under controlled storage conditions for about 15-30 days. The new tropical mango tree was asexually reproduced by grafting a scion of the tree to seedlings of the variety Turpentine.

### BRIEF DESCRIPTION OF THE DRAWINGS

The accompanying photographs show the tree and fruit of the present invention. In such photographs:

FIG. 1 shows a new tropical mango tree of the present invention;

FIG. 2 illustrates the foliage of the mango tree;

FIG. 3 shows a unripe mango;

FIG. 4 illustrates a mature mango; and

FIG. 5 shows the flesh of the mango.

### DETAILED BOTANICAL DESCRIPTION

The present invention relates to a new variety of tropical mango which was discovered growing in a cultivated area in North Miami Beach, Fla. The new mango tree is named “Leopold.”

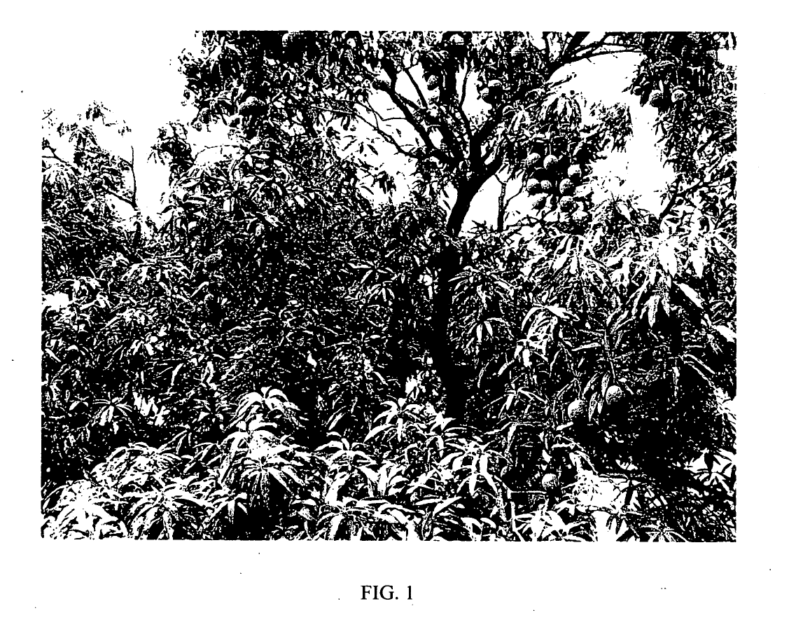

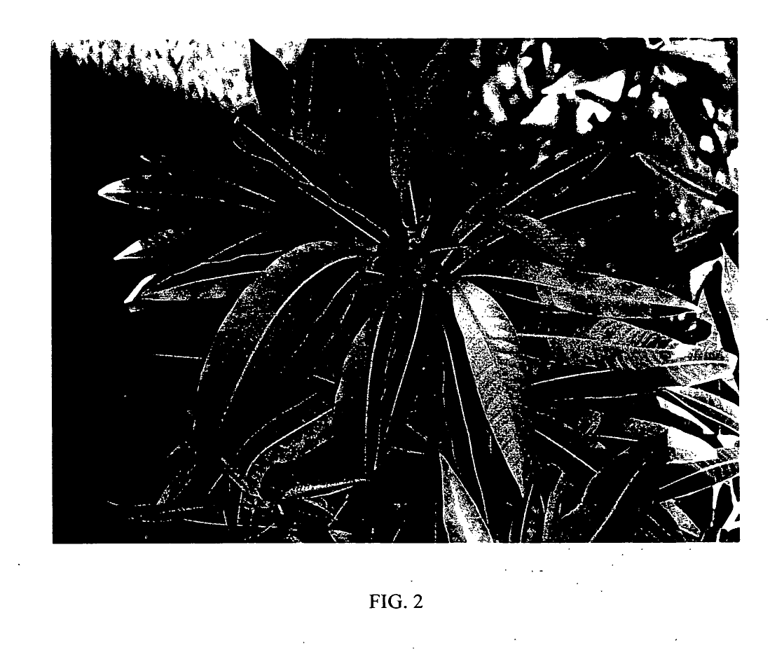

Referring now to the drawing figures, there is shown in FIG. 1 the mango tree of the present invention. This new mango variety has been asexually reproduced. Scions were grafted to Turpentine seedling trees. The characteristics that make the new mango a very desirable variety are the excellence of the fruit quality and its handling and storage ability. As shown in FIG. 2, the foliage of the new mango tree is of average density. The leaves are long and narrow with distinct veins. The leaves average between 6 to 14 inches in length and between 1 to 2½ inches in width. The leaves are dark green (Green Group 135B) when mature and are faded red (Greyed-Orange 177D) when new. The color codes 135B, 177D, and those hereinafter follow The Royal Horticultural Society's color numbering system.

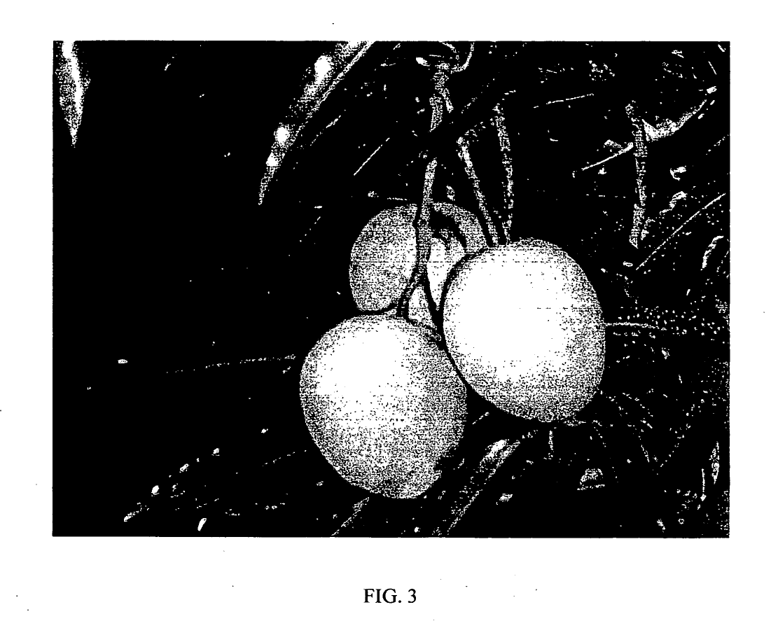

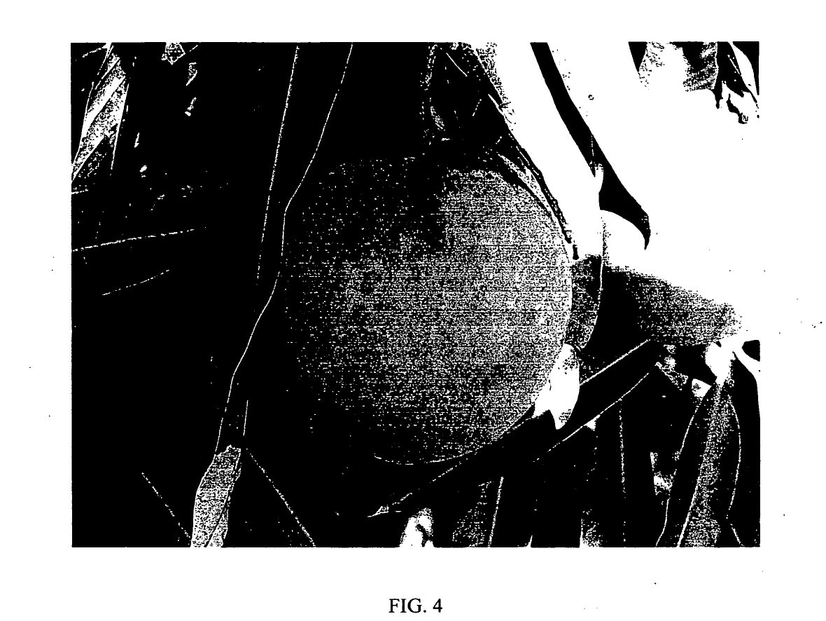

The mango tree blossoms in March, and its fruit matures in July and August. It bears a large plump fruit similar in shape to the mango named “Bombay Green.” The average weight of the new mango is 13 to 18 ounces when mature. As shown in FIG. 3, the new mango has a color in the Blue Green Group 133D. As the fruits mature, those exposed to direct sunlight take on a blush in the Purple Violet Group 80A, as shown in FIG. 4. A mature mango is approximately 3½ to 4½ inches in length and 2½ to 3½ inches in width.

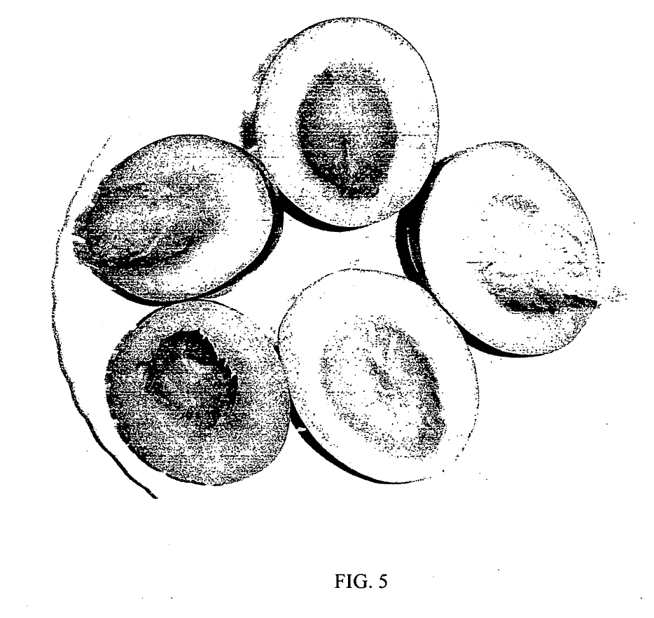

Referring now to FIG. 5, the flesh of the new mango is bright yellow to gold (Yellow-Orange Group 17C) with only a small amount of fiber attached to the husk of the seed. The seed is small in size, its shape is flat and is only a small percentage of total fruit weight, and the seed is freestone so that the fruit can be served cut in half and eaten with a spoon.

What is claimed is:

1. A new and distinct variety of tropical mango substantially as described and illustrated, having a mature fruit with good keeping qualities and which is freestone.
